(19) United States
(12) Patent Application Publication
(10) Pub. No.: US 2005/0267026 A1
(43) Pub. Date: Dec. 1, 2005

(54) Combination dosing regimen for erythropoietin

(76) Inventor: Wing K. Cheung, Warren, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/088,284

(22) Filed: Mar. 24, 2005

Related U.S. Application Data
(60) Provisional application No. 60/556,923, filed on Mar. 26, 2004.

(51) Int. Cl. A61K 38/18

(52) U.S. Cl. 514/12

(57) ABSTRACT

The present invention provides a combination dosing regimen for erythropoietin (EPO). More particularly, the present dosing regimen includes administration of at least a first dosing segment comprising a first exposure to EPO capable of stimulating the production of reticulocytes followed by a second exposure to EPO capable of sustaining the maturation of the reticulocytes into neocytes, and ultimately, red blood cells. Advantageously, the dosing segment may be cycled or repeated, any number of times and according to any desired time scheme, in order to provide or maintain any desired total red blood cell count and/or hemoglobin concentration. Methods of treatment employing the combination dosing regimen, as well as kits are also provided.

This Application claims priority from U.S. Provisional Application No. 60/556,923 entitled “Combination dosing regimen for erythropoietin” the contents of which are hereby incorporated by reference in their entirety.

### FIELD

The present invention provides a combination dosing regimen for erythropoietin effective to increase the production and maintenance of mature red blood cells and thereby also typically to increase hemoglobin concentration. Advantageously, the combination dosing regimen also can be cycled or repeated to maintain increases in hemoglobin concentration or to control hemoglobin concentration at the desired levels. Thus, the dosage regimen may be tailored to meet the particular needs of a variety of patients relative to conventional dosing schemes for EPO. As such, the invention also discloses methods of treatment are also provided, as are kits for carrying out the combination dosing regimen and methods.

### BACKGROUND

Oxygenation of the tissues and organs of the body is a complex process and relies upon efficient performance of several functions, e.g., oxygen uptake, delivery of oxygen to the tissues via oxygenated arterial blood, oxygen content of venous blood, etc. The inefficient performance of these functions as may occur due to any of a variety of causes, e.g., abnormal pulmonary function, arteriolar obstruction or vasoconstriction, or reduced concentration of hemoglobin, can result in insufficient tissue oxygenation, or tissue hypoxia.

Due to the numerous and complex functions involved, tissue oxygenation is indirectly assessable at best. Yet, treatment of tissue hypoxia is of utmost importance in the care of any patient since long term tissue hypoxia, also sometimes called anoxia, can result in irreversible tissue damage. A variety of treatments for tissue hypoxia have been developed, and although generally speaking, the optimal treatment for tissue hypoxia will be based at least in part upon the underlying cause, many patients with tissue hypoxia will derive some benefit from an increase in total red blood cell count, and the typically concurrent increase in hemoglobin associated therewith.

However, stimulation, production and maintenance of a desirable total red blood cell count are similarly complex undertakings. That is, every type of cell circulating in the blood system is derived from a pool of very primitive hematopoietic stem cells and have developed via any of a number of differentiation pathways. Thus, a limited number of cells are typically committed to any given pathway at any time. Once committed to a pathway, maturation and survival of these committed progenitors into a particular cell is not guaranteed, and further even if maturation is achieved, mature cells typically have a limited lifespan. Even if a greater percentage of stem cells can be caused to become committed progenitors to, e.g., the erythroid pathway that leads to mature red blood cells, only a certain number of these committed progenitors, or reticulocytes, will reach infant red blood cell stage, or become neocytes. Similarly, only a certain number of any such neocytes produced will actually survive to become mature red blood cells.

Conventional dosing schemes for EPO are illustrative of the difficulty of stimulating and maintaining increased red blood cell counts. That is, while such dosing schemes are effective to increase the concentration of reticulocytes circulating in the blood stream, they typically do not result in sustained increases, or any increase at all, in total red blood cell count and/or hemoglobin concentration. This result is not surprising when considered in light of the current understanding of the pharmacology of EPO; that EPO binds to receptors on committed progenitors to prevent apoptosis and to sustain the development of progenitors into reticulocytes.

For example, one conventional dosage scheme for EPO, e.g., a single subcutaneous injection of EPO, does indeed increase the concentration of reticulocytes in circulating blood as would be expected based upon conventional knowledge. However, data from subjects treated with this dosage scheme demonstrates that increases in the total red blood cell count and/or hemoglobin concentration are slight, transient, or both. A second conventional dosing scheme of weekly subcutaneous injections of the same dose of EPO can increase the total red blood cell count and result in enhanced hemoglobin concentration; however such a rigorous dosing regimen may be suboptimal in that it may discourage patient compliance and/or be cost prohibitive. Furthermore, such a dosing regimen does not provide any flexibility to tailor treatment according to an individual subjects' particular etiology or desired treatment outcome.

Desirably then, there would be provided a dosing regimen and/or method of treatment utilizing EPO that would cause an increase in the production of reticulocytes and also, would increase the number of reticulocytes that mature to neocytes, and further increase the number of these neocytes that survive to become mature red blood cells. Such a dosing regimen could provide further advantages if capable of maintaining any such increase for any desired or required length of time with a less rigorous, more flexible dosing schedule than weekly equivalent dosings.

### SUMMARY

The present invention provides a combination dosing regimen of EPO that provides such enhanced efficacy, while also providing more flexibility in dosing than conventional EPO dosing schemes. More particularly, the present dosing regimen can be effective to increase the production and maintenance of mature red blood cells, and thus, to increase hemoglobin concentration. Advantageously, the combination dosing regimen also can be cycled or repeated to maintain such increases, or indeed to provide a desired treatment outcome, so that the dosage regimen may be tailored to meet the particular needs of a wider variety of patients relative to conventional dosing schemes for EPO.

In a first aspect, the present invention thus provides a dosing regimen of EPO. The dosing regimen comprises administration of at least a first dosing segment comprising a first exposure to EPO effective to at least marginally increase production of reticulocytes followed by a second exposure to EPO at least marginally effective to sustain the reticulocytes as they mature into red blood cells. Desirably, administration of the second exposure is initiated within about 3 days but not more than about 10 days after the first exposure. Each dosing segment thus has the effect of increasing total red blood cell count and/or hemoglobin concentration. Additionally, the dosing segment can be repeated, or cycled, as many times as desired or required, in order to maintain, or adjust, the total red blood cell count or hemoglobin level. Further, the first and second exposures may comprise any dosing form and any dosage amount, and may be the same or different.

Unlike conventional dosing schemes, the present inventive dosing scheme can not only provide a significant initial increase in total red blood cell counts and/or hemoglobin concentration, but also can be used to sustain the same at any desired level. Advantageously, the present dosing regimen does so while also providing the flexibility heretofore lacking in EPO administration and indeed can be tailored to meet the particular needs of any given subject. The dosing regimen thus finds application in the treatment of any subject needing or desiring an increase and maintenance in total red blood cell count and/or hemoglobin concentration.

In a second aspect then, the present invention provides a method for enhancing the production and maintenance of mature red blood cells, and as a result, typically also increasing hemoglobin concentration. More particularly, the method comprises administering at least a first dosing segment comprising a first exposure to EPO effective to at least marginally increase production of reticulocytes followed by a second exposure to EPO effective to demonstrate at least some increase in the ability of the reticulocytes to mature into red blood cells. Administration of the second exposure to EPO is desirably initiated within about 3 days but not more than about 10 days after the first exposure, in order exposure EPO to any neocytes formed via the first exposure at the time the neocytes are expected to mature into red blood cells. In certain embodiments, it may be desirable to provide newly developed neocytes with a sustained exposure to EPO for example, using multiple low daily doses, an EPO implant or patch, or a long-acting EPO.

The dosing segment may then advantageously be repeated at any desired interval to provide any desired result, as may be determined by a subject's particular needs, e.g., at intervals timed to substantially maintain, increase or decrease red blood cell counts. Such management of red blood cell counts and/or hemoglobin concentration can be advantageous to a variety of subjects, including but not limited to, those suffering from tissue hypoxia arising from any cause, e.g., anemia or chronic anemia, subjects receiving chemotherapy, and subjects that have suffered traumatic injury. The present inventive methods may be employed to treat any of these.

The components for carrying out the present dosing regimen and method, i.e., dosing forms of EPO, are readily commercially available. However, to enhance the convenience, flexibility, availability and usability of the dosing regimen and methods, the components required to practice the inventive regimen and methods may be provided in conjunction with one another in the form of a kit, and such a kit is contemplated to be within the scope of the present invention.

The inventive kit would desirably comprise at least a first dosing segment of EPO comprising a first dosing unit of EPO capable of providing an exposure to EPO effective to at least marginally increase production of reticulocytes. A second dosing unit of EPO, comprising a different dosage amount or form of EPO than the first and capable of providing an exposure to EPO at least marginally effective to sustain the reticulocytes as they mature into red blood cells. The kit may further include instructions indicating that administration of the second exposure is to be initiated within about 3 days but not more than about 10 days after the first exposure. In certain embodiments, the instructions may further indicate that the dosing segment may be repeated, e.g., at least about 2 weeks, or at least about 4 weeks, or even at least about 8 weeks after administration of the preceding segment. The instructions in certain embodiments of the kit may also be tailored to particular subjects, e.g., for subjects undergoing chemotherapy, the instructions may indicate that the dosing segment may desirably be repeated in concert with chemotherapy treatments.

### BRIEF DESCRIPTION OF THE FIGURES

The accompanying figures illustrate several aspects of the invention. A brief description of the figures is as follows:

FIG. 1 is a graphical depiction of the conventionally understood pharmacology of EPO, i.e., that administration of a single subcutaneous dose of EPO in healthy subjects produces a mean percent increase in reticulocyte levels that can be directly related to the size of the dose of EPO administered (in terms of area under the concentration-time curve of % reticulocytes);

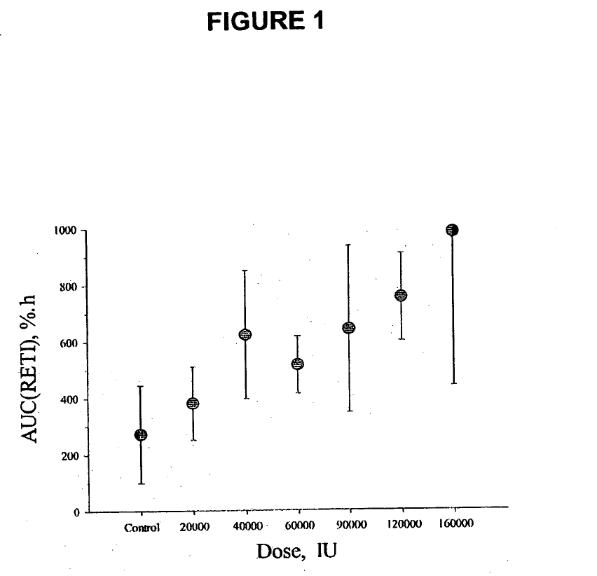

FIG. 2 is a graphical depiction of mean serum EPO levels in healthy subjects after a conventional dosing of EPO, e.g., a single subcutaneous dose of 40,000 IU EPO, showing a peak upon dosing thereafter returning to endogenous levels within about 10 days;

FIG. 3 is a graphical depiction of mean percent reticulocyte levels in healthy individuals after administration of the same conventional dose of EPO depicted in FIG. 2, showing a peak in reticulocyte levels about 7 days after a single subcutaneous administration of EPO, thereafter returning to pre-dose levels within about 15 days;

FIG. 4 is a graphical depiction of mean red blood cell count in healthy individuals after administration of the same conventional dose of EPO depicted in FIG. 2, showing that no significant increase in red blood cell count is seen upon or after conventional dosing with EPO;

FIG. 5 is a graphical depiction of mean hemoglobin levels in healthy individuals after administration of the same conventional dose of EPO depicted in FIG. 2, showing that this conventional dosing with EPO does not produce a significant or sustained increase in hemoglobin levels;

FIG. 6 is a graphical depiction comparing serum EPO levels after (i) conventional dosing with EPO, i.e., administration of a single subcutaneous 400 IU/kg dose of EPO, (ii) low level daily EPO injections of 25 IU/kg/day, and (iii) dosing in accordance with one embodiment of a dosing segment as contemplated by the present invention, i.e., a first exposure to 400 IU/kg EPO followed by a second exposure to 25 IU/kg/day EPO on days 4-16 after the first exposure, showing that all dosing schemes produced a peak in EPO upon dosing thereafter returning to endogenous levels within about 5 days;

FIG. 7 is a graphical depiction comparing reticulocyte levels after (i) conventional dosing with EPO, i.e., administration of a single subcutaneous 400 IU/kg dose of EPO, (ii) low level daily EPO injections of 25 IU/kg/day, and (iii) dosing in accordance with one embodiment of a dosing segment as contemplated by the present invention, i.e., a first exposure to 400 IU/kg EPO followed by a second exposure to 25 IU/kg/day EPO on days 4-16 after the first exposure, showing that all dosing schemes produced a peak in reticulocytes upon dosing thereafter returning to endogenous levels within about 30 days;

FIG. 8 is a graphical depiction comparing changes in red blood cell levels after (i) conventional dosing with EPO, i.e., administration of a single subcutaneous 400 IU/kg dose of EPO, (ii) low level daily injections of 25 IU/kg/day EPO, and (iii) dosing in accordance with one embodiment of a dosing segment as contemplated by the present invention, i.e., a first exposure to 400 IU/kg EPO followed by a second exposure to 25 IU/kg/day EPO on days 4-16 after the first exposure, showing that dosing according to this embodiment of the dosing segment produced a significant increase in red blood cell count relative to both conventional dosing and low level daily dosing with EPO;

FIG. 9 is a graphical depiction comparing change in hemoglobin levels after (i) conventional dosing with EPO, i.e., administration of a single subcutaneous 400 IU/kg dose of EPO, (ii) low level daily injections of 25 IU/kg/day EPO, and (iii) dosing in accordance with one embodiment of a dosing segment as contemplated by the present invention, i.e., a first exposure to 400 IU/kg EPO followed by a second exposure to 25 IU/kg/day EPO on days 4-16 after the first exposure, showing that dosing according to this embodiment of the dosing segment produced a significant and sustained increase in hemoglobin relative to both conventional dosing and low level daily dosing with EPO;

FIG. 10 is a graphical depiction comparing serum EPO levels after (i) conventional dosing with EPO, i.e., administration of a single subcutaneous 400 IU/kg dose of EPO, (ii) low level daily EPO injections of 50 IU/kg/day, and (iii) dosing in accordance with one embodiment of a dosing segment as contemplated by the present invention, i.e., a first exposure to 400 IU/kg EPO followed by a second exposure to 50 IU/kg/day EPO on days 4-16 after the first exposure, showing that all dosing schemes produced a peak in EPO upon dosing thereafter returning to endogenous levels within about 5-7 days;

FIG. 11 is a graphical depiction comparing reticulocyte levels after (i) conventional dosing with EPO, i.e., administration of a single subcutaneous 400 IU/kg dose of EPO, (ii) low level daily EPO injections of 50 IU/kg/day, and (iii) dosing in accordance with one embodiment of a dosing segment as contemplated by the present invention, i.e., a first exposure to 400 IU/kg EPO followed by a second exposure to 50 IU/kg/day EPO on days 4-16 after the first exposure, showing that all dosing schemes produced a peak in reticulocytes upon dosing thereafter returning to endogenous levels within about 30 days;

FIG. 12 is a graphical depiction comparing changes in red blood cell levels after (i) conventional dosing with EPO, i.e., administration of a single subcutaneous 400 IU/kg dose of EPO (ii) low level daily EPO injections of 50 IU/kg/day, and (iii) dosing in accordance with one embodiment of a dosing segment as contemplated by the present invention, i.e., a first exposure to 400 IU/kg EPO followed by a second exposure to 50 IU/kg/day EPO on days 4-16 after the first exposure, showing that dosing according to this embodiment of the dosing segment produced a significant increase in red blood cell count relative to both conventional dosing and low level daily dosing of EPO;

FIG. 13 is a graphical depiction comparing the change in hemoglobin levels after (i) conventional dosing with EPO, i.e., administration of a single subcutaneous 400 IU/kg dose of EPO, (ii) low level daily EPO injections of 50 IU/kg/day, and (iii) dosing in accordance with one embodiment of a dosing segment as contemplated by the present invention, i.e., a first exposure to 400 IU/kg EPO followed by a second exposure to 50 IU/kg/day EPO on days 4-16 after the first exposure, showing that dosing according to this embodiment of the dosing segment produced a significant increase in hemoglobin relative to both conventional dosing and low level daily dosing with EPO;

FIG. 14 is a graphical depiction comparing serum EPO levels after (i) conventional dosing with EPO, i.e., administration of a single subcutaneous 800 IU/kg dose of EPO, (ii) low level daily. EPO injections of 25 IU/kg/day, and (iii) dosing in accordance with one embodiment of a dosing segment as contemplated by the present invention, i.e., a first exposure to 800 IU/kg followed by a second exposure to 25 IU/kg/day EPO on days 4-16 after the first exposure, with all dosing schemes producing a peak in EPO upon dosing thereafter returning to endogenous levels within about 5-7 days;

FIG. 15 is a graphical depiction comparing reticulocyte levels after (i) conventional dosing with EPO, i.e., administration of a single subcutaneous 800 IU/kg dose of EPO, (ii) low level daily EPO injections of 25 IU/kg/day, and (iii) dosing in accordance with one embodiment of a dosing segment as contemplated by the present invention, i.e., a first exposure to 800 IU/kg followed by a second exposure to 25 IU/kg/day EPO on days 4-16 after the first exposure, with all dosing schemes producing a peak in reticulocytes upon dosing thereafter returning to endogenous levels within about 30 days;

FIG. 16 is a graphical depiction comparing changes in red blood cell levels after (i) conventional dosing with EPO, i.e., administration of a single subcutaneous 800 IU/kg dose of EPO, (ii) low level daily EPO injections of 25 IU/kg/day, and (iii) dosing in accordance with one embodiment of a dosing segment as contemplated by the present invention, i.e., a first exposure to 800 IU/kg followed by a second exposure to 25 IU/kg/day EPO on days 4-16 after the first exposure, showing that dosing according to this embodiment of the dosing segment produced a significant increase in red blood cell count relative to both conventional dosing and low level daily dosing of EPO;

FIG. 17 is a graphical depiction comparing changes in hemoglobin levels after (i) conventional dosing with EPO, i.e., administration of a single subcutaneous 800 IU/kg dose of EPO, (ii) low level daily EPO injections of 25 IU/kg/day, and (iii) dosing in accordance with one embodiment of a dosing segment as contemplated by the present invention, i.e., a first exposure to 800 IU/kg followed by a second exposure of 25 IU/kg/day EPO on days 4-16 after the first exposure, showing that dosing according to this embodiment of the dosing segment produced a significant increase in hemoglobin relative to both conventional dosing and low level daily dosing with EPO;

FIG. 18 is a graphical depiction comparing serum EPO levels after (i) conventional dosing with EPO, i.e., administration of a single subcutaneous 800 IU/kg dose of EPO, (ii) low level daily EPO injections of 50 IU/kg/day, and (iii) dosing in accordance with one embodiment of a dosing segment as contemplated by the present invention, i.e., a first exposure to 800 IU/kg following by a second exposure to 50 IU/kg/day EPO on days 4-16 after the first exposure, showing that all dosing schemes produced a peak in EPO upon dosing thereafter returning to endogenous levels within about 5-7 days;

FIG. 19 is a graphical depiction comparing reticulocyte levels after (i) conventional dosing with EPO, i.e., administration of a single subcutaneous 800 IU/kg dose of EPO, (ii) low level daily EPO injections of 50 IU/kg/day, and (iii) dosing in accordance with one embodiment of a dosing segment as contemplated by the present invention, i.e., a first exposure to 800 IU/kg following by a second exposure to 50 IU/kg/day EPO on days 4-16 after the first exposure, showing that all dosing schemes produced a peak in reticulocytes upon dosing, and further, that dosing according to this embodiment of the dosing segment produced a second peak in reticulocyte levels at about 30 days;

FIG. 20 is a graphical depiction comparing changes in red blood cell levels after (i) conventional dosing with EPO, i.e., administration of a single subcutaneous 800 IU/kg dose of EPO, (ii) low level daily EPO injections of 50 IU/kg/day, and (iii) dosing in accordance with one embodiment of a dosing segment as contemplated by the present invention, i.e., a first exposure to 800 IU/kg following by a second exposure to 50 IU/kg/day EPO on days 4-16 after the first exposure, showing that dosing according to this embodiment of the dosing segment produced a significant increase in red blood cell count relative to both conventional dosing and low level daily dosing of EPO;

FIG. 21 is a graphical depiction comparing changes in hemoglobin levels after (i) conventional dosing with EPO, i.e., administration of a single subcutaneous 800 IU/kg dose of EPO, (ii) low level daily EPO injections of 50 IU/kg/day, and (iii) dosing in accordance with one embodiment of a dosing segment as contemplated by the present invention, i.e., a first exposure to 800 IU/kg following by a second exposure to 50 IU/kg/day EPO on days 4-16 after the first exposure, showing that dosing according to this embodiment of the dosing segment produced a significant increase in hemoglobin relative to both conventional dosing and low level daily dosing with EPO; and

FIG. 22 is a graphical depiction showing the change in hemoglobin levels after (i) conventional dosing with EPO, i.e., administration of subcutaneous 600 IU/kg doses of EPO weekly for 4 weeks, (ii) 1800 IU/kg EPO at week 5 and again at week 8 and (iii) dosing in accordance with one embodiment of the inventive combination dosing regimen, i.e., a dosing segment of a first exposure to 600 IU/kg EPO on week 5 and a second exposure to 1200 IU/kg on week 6, repeating the dosing segment at weeks 8 (600 IU/kg EPO) and 9 (1200 IU kg), showing that while hemoglobin started declining on Week 6 for those conventionally dosed, hemoglobin was maintained in an elevated state for those dosed according to the present combination dosing regimen.

### DETAILED DESCRIPTION

The embodiments of the present invention described below are not intended to be exhaustive or to limit the invention to the particular embodiments disclosed in the following detailed description. Rather, the embodiments are described so that others skilled in the art can understand the principles and practices of the present invention. If not specifically mentioned below, the disclosures of each patent, published patent application and publication referenced in the following description are hereby incorporated by reference in their entirety for any and all purposes.

The present invention provides a combination dosing regimen for EPO that not only can stimulate production of reticulocytes, but also that can surprisingly sustain the neocytes that develop therefrom so that a substantial portion of the neocytes mature into red blood cells. As such, the present dosing regimen can provide an initial increase in total red blood cell count and hemoglobin concentration greater than that achieved via administration of a single conventional dose of EPO. Furthermore, the present combination dosing regimen can be cycled, or repeated, to substantially maintain this initial increase surprisingly not via a more rigorous dosing schedule than conventional once per week dosing, but in fact, with a greater length of time between dosing segments. Indeed, the newly discovered pharmacological functionality of EPO disclosed herein may be taken advantage of in a wide variety of ways, thereby providing a tremendous amount of flexibility to a health care provider to tailor treatment according to the present dosing combination to fit any particular subject's needs or desires.

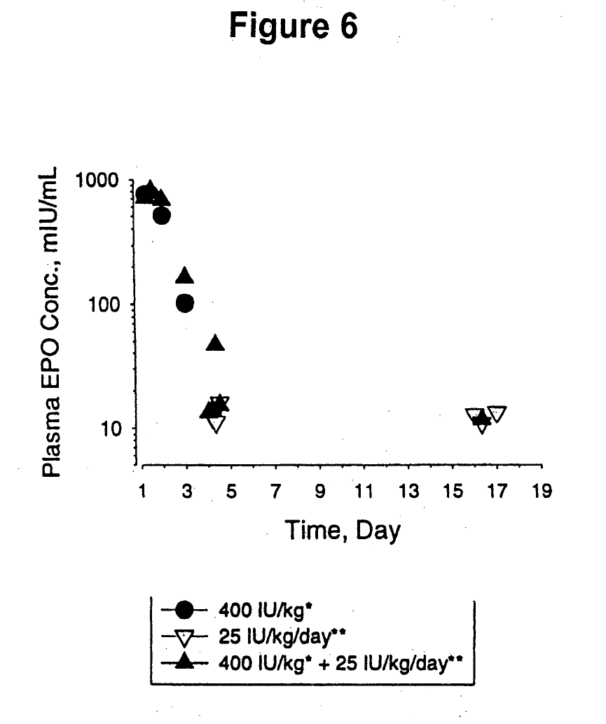

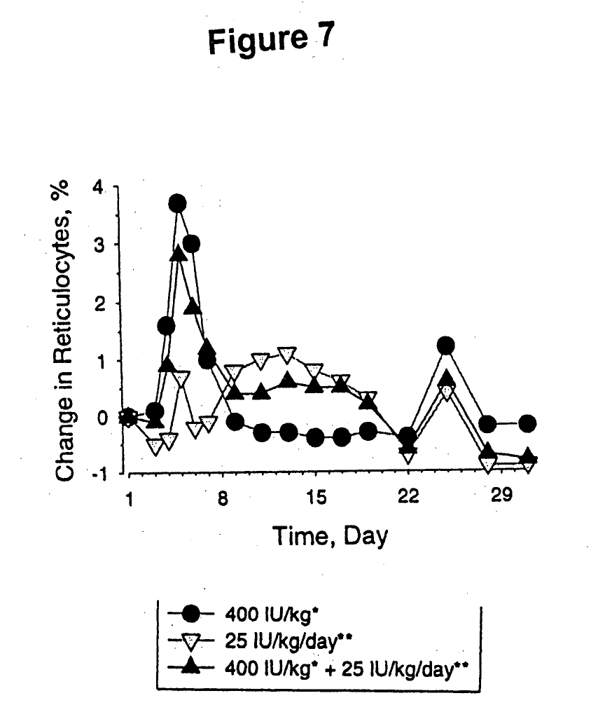

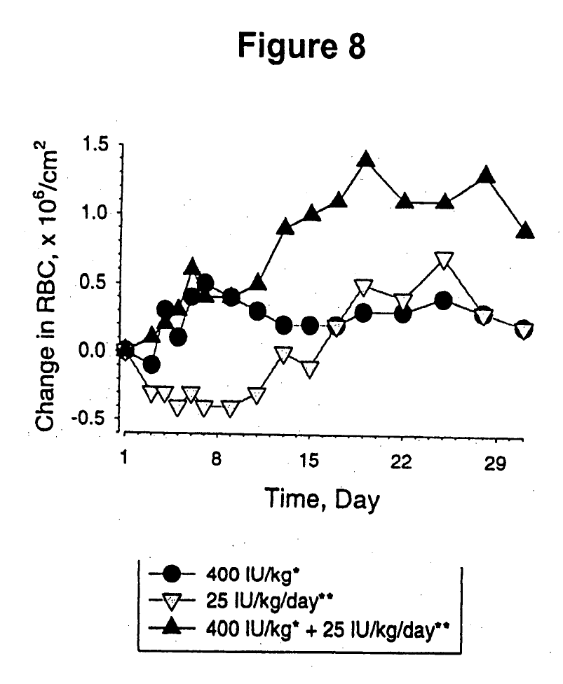

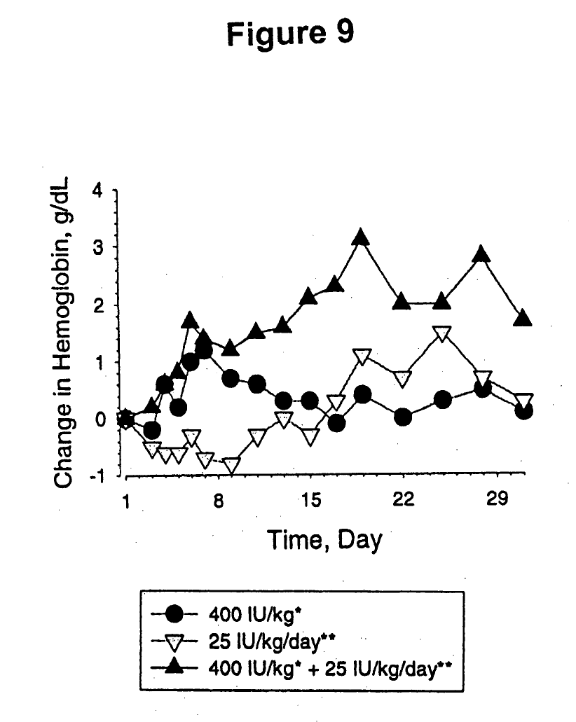

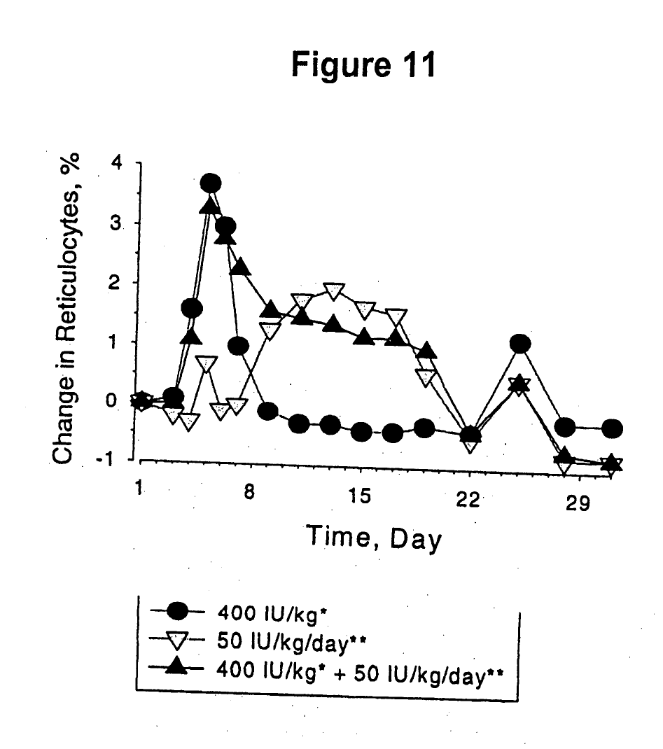

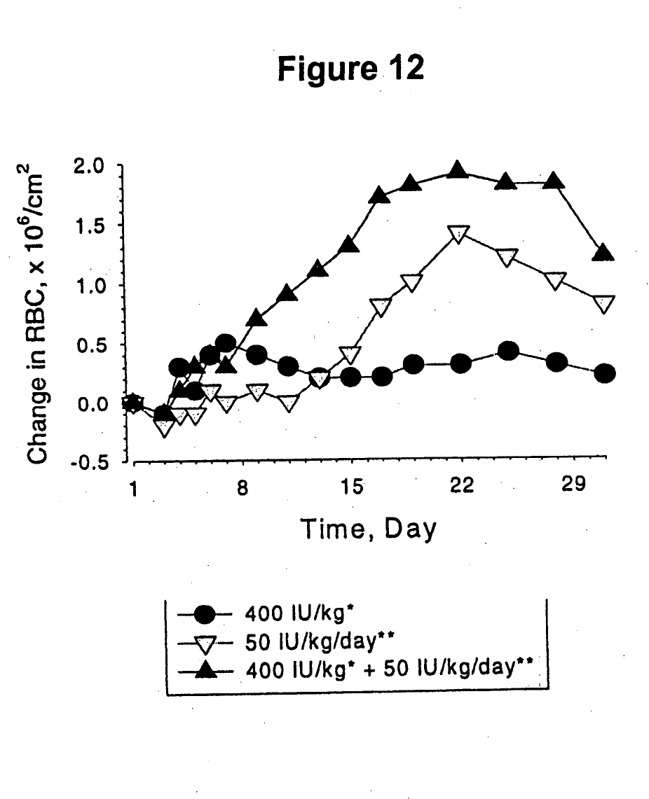

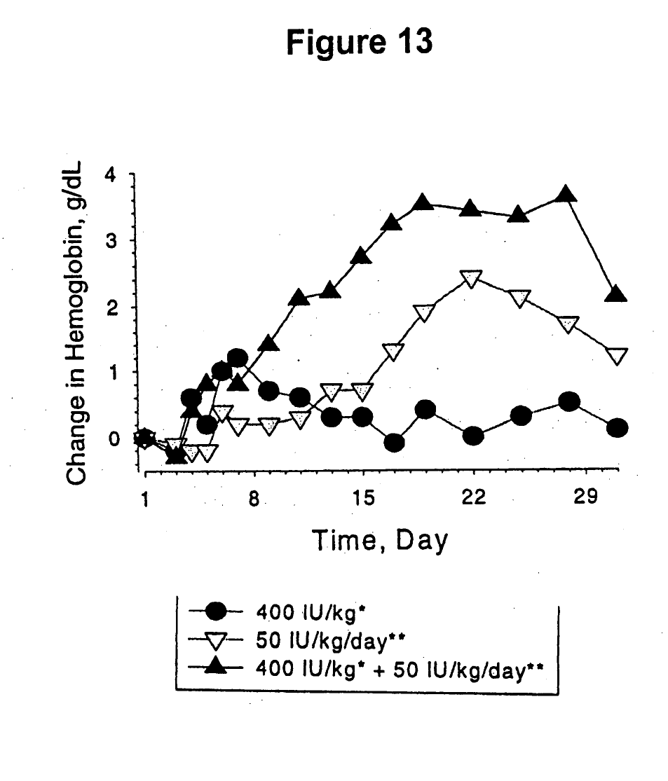

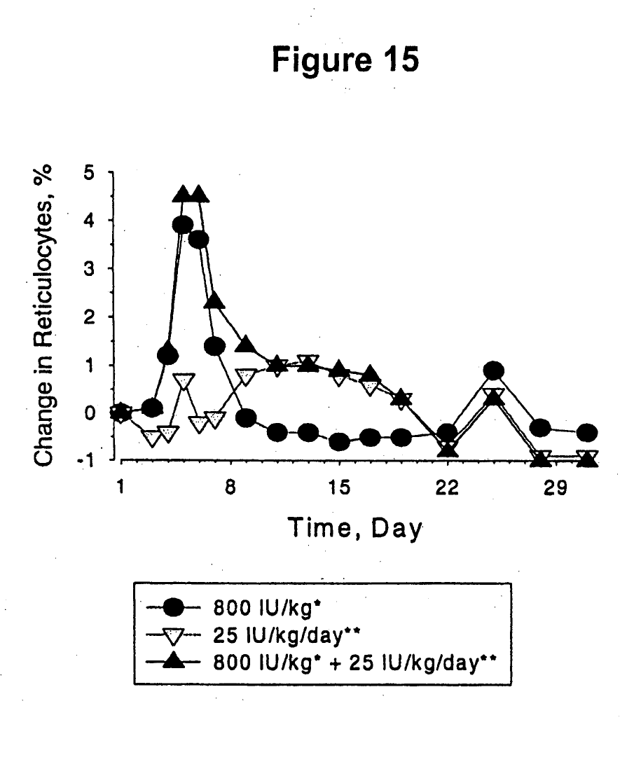

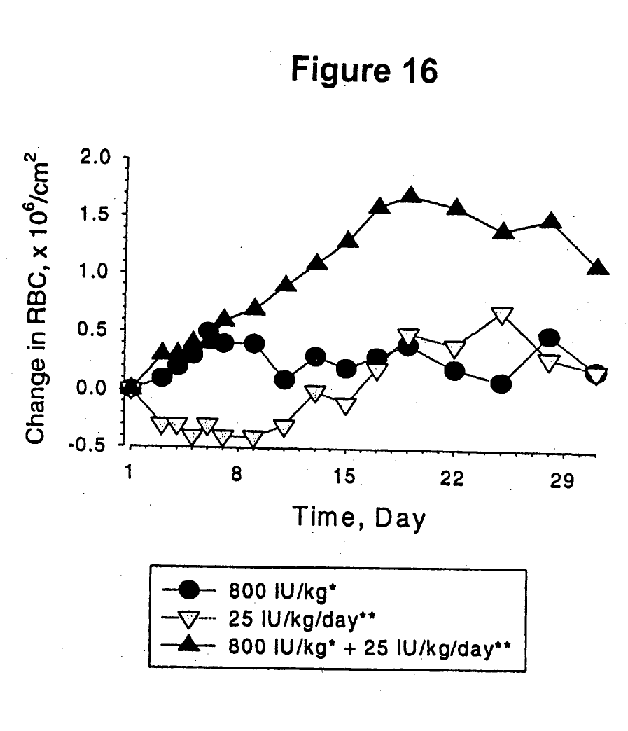

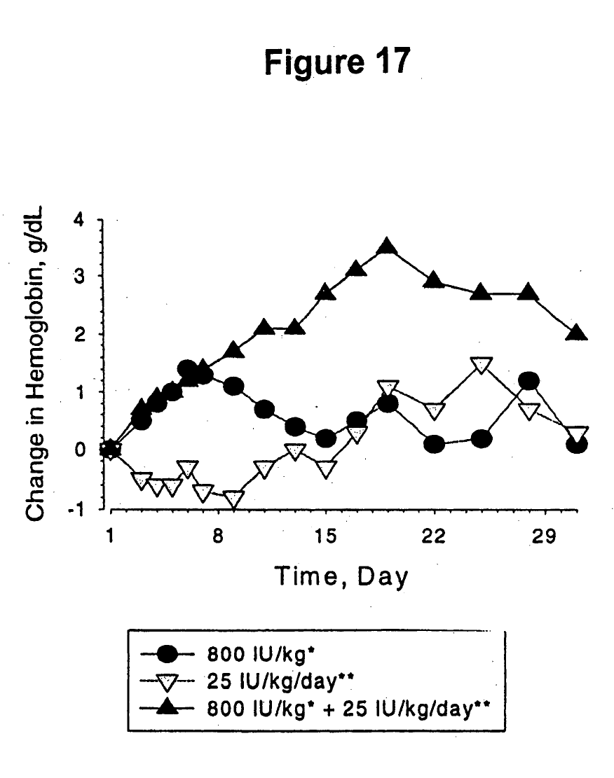

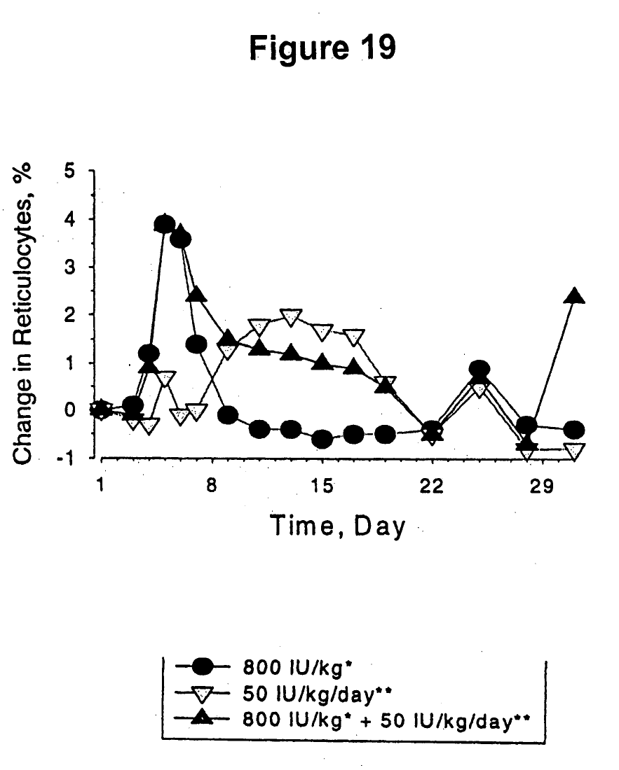

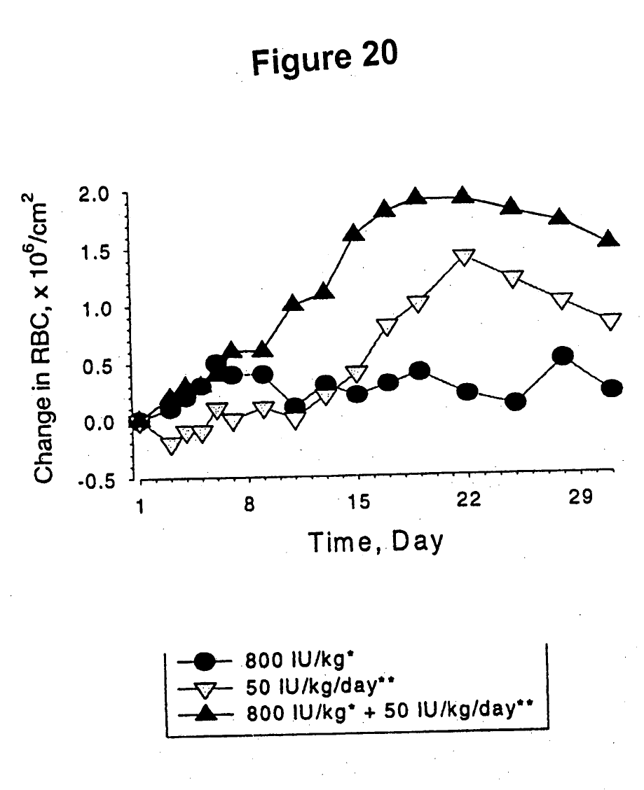

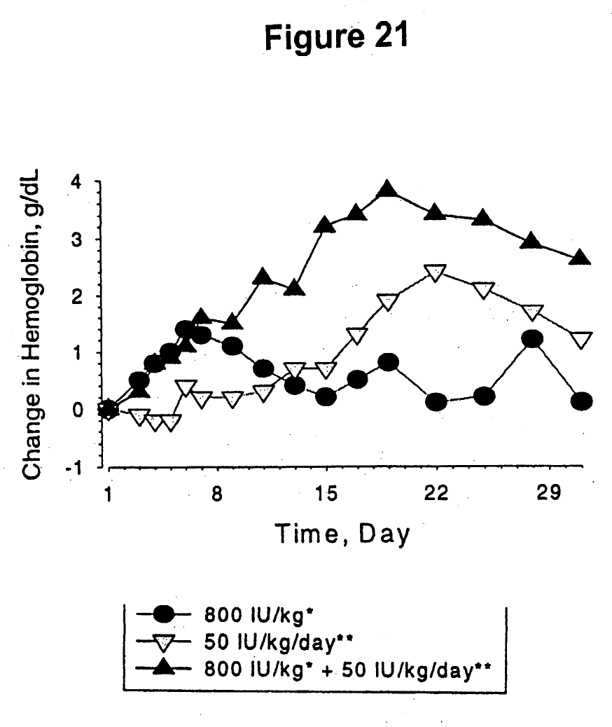

More particularly, Applicants have now surprisingly discovered that EPO appears to at least somewhat assist in the sustenance of neocytes as they mature into red blood cells. Although not supported by the conventional understanding of the pharmacological activity of EPO, Applicants nonetheless have compelling data supporting this newly discovered activity, some of which is shown in the FIGS. 6-22 of the present application. Indeed, conventional pharmacologic understanding provides no basis for this newly discovered activity since there are not currently understood to be any EPO receptors on reticulocytes or neocytes, making it all the more surprising and unexpected. Taking advantage of this newly discovered activity provides many new avenues for the therapeutic use of EPO, and any such use, relying upon the episodic or continual exposure of reticulocytes and neocytes to EPO for the enhancement of their survival and maturation into mature red blood cells, is considered to be within the scope of the present invention.

One exemplary advantage that can be provided via dosing in accordance with this new discovery, i.e., in support of the sustenance and maturation of reticulocytes and neocytes, is that the increases in total red blood cell counts and/or hemoglobin concentration that can be provided thereby can be maintained, or otherwise managed, via much more flexible, and less rigorous dosing schemes than conventional dosing schemes for EPO. Conventional dosing schemes, relying on conventional pharmacology suggests that EPO acts to stimulate the dedication of progenitors to the erythroid cycle. The newly discovered activity of EPO, coupled with the knowledge that mature red blood cells typically live about 120 days, allows for a much less rigorous dosing of EPO, while providing similar, or even better increases in total red blood cell count and/or hemoglobin concentration.

The present invention thus provides a combination dosing regimen of EPO that, via administration of at least one dosing segment, stimulates dedication of progenitors to become reticulocytes and neocytes and then assists in the sustenance and maturation thereof as they develop into mature red blood cells. The term “dosing segment” refers to a dosing schedule that includes an initial exposure to EPO where the exposure is effective to stimulate a measurable increase in the production of reticulocytes, however slight that measurable increase might be, followed by a second exposure effective to sustain the reticulocytes and neocytes and to assist in the maturation thereof into mature red blood cells. Since reticulocytes, once introduced into the blood stream, are expected to mature into neocytes in about 2 days, and neocytes are believed to begin the maturation process beginning at least about 10-15 days after introduction of reticulocytes into the bloodstream, the second exposure is desirably and most advantageously initiated about 3 to about 10 days after the cessation of the first exposure. Further, since it is further believed that exposure to EPO, be it native EPO or exogenously administered EPO, may be required in order to sustain the survival of the neocytes during this maturation period, the second exposure is desirably and most advantageously initiated no more than about 10 days after the cessation of the first exposure, and may further advantageously be sustained, either by repeated administration or administration of a long-acting EPO, or other continued exposure to EPO, i.e., such as via patch, or implant, for at least about 7 days and for up to at least about 15 days.

As used herein, the term “exposure” is meant to indicate a single dose, repeated individual doses, or dosing as may be provided relatively continuously after a single administration, e.g., of a long-acting EPO, application, e.g., of a transdermal patch comprising EPO, or implantation of an EPO implant. That is, the particular mode of administration of the dose, and indeed the amount thereof, is not critical to the practice of the present dosing segment or regimen. All that is required is that a first exposure be administered in any amount and any fashion that is effective to stimulate the production of reticulocytes, followed by administration of a second exposure in any amount and any fashion effective to enhance the sustenance and maturation of reticulocytes and neocytes into mature red blood cells. Further, any mode of administration may be utilized for either exposure, including those currently known and those that may be developed in the future. Moreover, the term “exposure” refers to the administration of a dose of EPO to a patient to be distinguished from exposures that a patient may have to their own native levels of endogenous EPO.

Those of ordinary skill in the art are readily capable of determining appropriate amounts and manners of exposure in either instance. That being said, and only for the purpose of providing additional guidance and not being unnecessarily bound thereto, general therapeutic guidelines suggest that subjects would desirably have a hemoglobin concentration above at least about 9 g/dL, and dosages of EPO capable of providing such a hemoglobin level are appropriate and may be readily and easily determined by those of ordinary skill in the art. The art generally understands that elevated levels of hemoglobin above the current clinical medicine norms may be associated in some cases with increased risk of thrombovascular events to the patient. Therefore, those skilled in the art will appreciate that hemoglobin levels should be monitored regularly throughout any EPO treatment regime.

As such, the amount of EPO to be administered in any particular exposure of any given dosing segment is limited only by patient safety concerns and any amount of EPO may be administered per exposure, dosing segment, or combination dosing regimen so long as substantially no toxic effects due to administration of EPO are manifested. However, it is believed that although stimulation of reticulocytes upon administration of the first exposure of a dose segment is dose responsive, dose saturation may be attained at doses greater than about 2400 IU/kg. Thus, while no harm may be done, no further benefit may be seen if a first exposure is administered in excess of this amount. If only for economic purposes then, the first exposure of EPO in a dosing segment may provide from about 150 IU/kg to about 2400 IU/kg EPO, or preferably, from about 600 IU/kg to about 1200 IU/kg EPO. As mentioned above, the exposure can be provided in any known, or newly developed, dosing format. That being said, since the desired outcome of the first exposure is stimulation of the maximum amount of reticulocytes and since such stimulation is believed to be dose dependent, the first exposure of EPO may desirably be provided in a format that can provide the exposure substantially instantaneously, e.g., such as, by injection, whether it be subcutaneous, intramuscularly, or intraperitonealy.

Similarly, and although it is believed that the greater the second exposure of EPO administered in a dosing segment, the longer the duration of elevation of EPO levels in the blood and the greater the impact on the survival and maturation rate of neocytes into red blood cells, dose saturation is expected to occur at exposures that provide greater than about 2400 IU/kg EPO. Suitable amounts of EPO for administration as a second exposure of a dose segment thus can be from about 600 IU/kg to about 2400 IU/kg EPO, or preferably from about 900 IU/kg to about 1500 IU/kg EPO.

As mentioned above, Applicants have now surprisingly discovered that this second exposure of EPO, timed in administration to coincide with the maturation of reticulocytes and/or neocytes into mature red blood cells, can assist them in doing the same. Applicants research further suggests that the length of exposure at this stage may be as, or even more, important than the overall size of the dose itself to which the reticulocytes/neocytes are exposed. As such, the second exposure may advantageously and desirably be provided as multiple low level exposures, e.g., of less than about 100 IU/kg/day, or less than about 50 IU/kg/day, or even less than about 25 IU/kg/day, as may be provided by, e.g., by subcutaneous injections, a transdermal patch, an implant, administration of a long-acting EPO, and the like. Many long-acting EPO's are known and are commercially available and any of these may be used.

Each dosing segment administered in accordance with the present combination dosing regimen can provide a dose-dependent increase in the reticulocyte concentration via the first exposure as may be expected based upon conventional pharmacological understanding of EPO. Each dosing segment surprisingly may also assist in the sustenance and maturation of a substantial percentage of the reticulocytes and neocytes into mature red blood cells, a result which is not expected, predictable based upon conventional wisdom, nor indeed seen after a conventional dose, e.g., a single subcutaneous, dose of EPO.

Although advantageous in and of itself, this enhanced maturation rate of neocytes can be further exploited to provide yet additional advantages. That is, since the enhanced maturation of neocytes can produce increased numbers of mature red blood cells, there is more flexibility in dosing EPO to maintain the mature red blood cells. This is in contrast to current regimens that focus on the use of large weekly doses of EPO to continuously stimulate reticulocytes. Also, if an increase or decrease in total red blood cell count and/or hemoglobin concentration is desired or required, administration of the dosing segment or segments may be varied accordingly. As such, the combination dosing regimen of the present invention can be tailored to a subject's particular needs or desires.

Advantageously then, the dosing segment may be repeated after a time interval appropriate to effectuate the desired outcome, or after at least about 2 weeks, or even about 4 weeks, and in fact, repetition of the dosing segment after about 8 weeks may be sufficient in some instances to maintain the increase in total red blood cell count and/or hemoglobin provided by the immediately preceding dosing segment. Similarly, the dosing segment can be repeated as many times as desired or required to provide a combination dosing regimen capable of spanning any desired treatment period and the number of repetitions is not restricted in any fashion.

As used herein, “erythropoietin” or “EPO” shall include those polypeptides and proteins that have the biological activity of recombinant human erythropoietin (r-HuEPO), that is, they bind to the EPO receptor and ultimately stimulate an increase in hemoglobin production. The term EPO, as used herein also refers to erythropoietin analogs, erythropoietin isoforms, erythropoietin mimetics, erythropoietin fragments, hybrid erythropoietin proteins, fusion proteins, oligomers and multimers of the above, homologues of the above, glycosylation pattern variants of the above, including pegylated EPO, muteins of the above, and further regardless of the method of synthesis or manufacture thereof including, but not limited to, recombinant (whether produced from cDNA or genomic DNA), synthetic, transgenic, and gene activated methods, and further those EPO molecules containing the minor modifications enumerated above. Methods of designing and synthesizing, e.g., peptide mimetics are well known to those of ordinary skill in the art and are described, e.g., in U.S. Pat. Nos. 4,833,092, 4,859,765; 4,853,871 and 4,863,857 the disclosures of each of which are hereby incorporated by reference herein in their entirety and for all purposes.

Particularly preferred EPO molecules are those that are capable of stimulating erythropoiesis in a mammal. Specific examples of erythropoietin include, Epoetin alfa (EPREX®, ERYPO®, PROCRIT®), as well as erythropoiesis stimulating protein (NESP™, ARANESP™, darbepoetin alfa) and hyperglycosylated analog of recombinant human erythropoietin (Epoetin) such as those described in European patent application EP640619, as well as CERA™, human erythropoietin analogs (such as the human serum albumin fusion proteins described in the international patent application WO 99/66054), erythropoietin mutants described in the international patent application WO 99/38890, erythropoietin omega, which may be produced from an Apa I restriction fragment of the human erythropoietin gene described in U.S. Pat. No. 5,688,679, altered glycosylated human erythropoietin described in the international patent application WO 99/11781 and EP 1064951, and PEG conjugated erythropoietin analogs described in WO 98/05363, WO 01/76640, or U.S. Pat. No. 5,643,575. Specific examples of cell lines modified for the expression of endogenous human erythropoietin are described in international patent applications WO 99/05268 and WO 94/12650. A preferred form of EPO is purified recombinant human EPO (r-HuEPO), currently formulated and distributed under the trademarks of EPREX®, ERYPO®, PROCRIT® or ARANESP™. The disclosures of each of the patents and published patent applications mentioned in this paragraph are hereby incorporated by reference herein for any and all purposes.

Long-acting forms of EPO are also contemplated, and indeed, may be preferred in some embodiments of the present invention for administration as the second exposure in a dosing segment. As used herein, a “long-acting EPO” includes sustained-release compositions and formulations of EPO with increased circulating half-life, typically achieved through modifications which reduce immunogenicity or alter clearance rate. Also included is EPO encapsulated in polymer microspheres. Examples of “long-acting EPO” include, but are not limited to, conjugates of erythropoietin with polyethylene glycol (PEG) disclosed in PCT publication WO 2002049673 (Burg et al.), PEG-modified EPO disclosed in PCT publication WO 02/32957 (Nakamura et al.), conjugates of glycoproteins having erythropoietic activity and having at least one oxidized carbohydrate moiety covalently linked to a non-antigenic polymer disclosed in PCT publication WO 94/28024 (Chyi et al.), and other PEG-EPO molecules prepared using SCM-PEG, SPA-PEG AND SBA-PEG. The disclosures of each of these published patent applications are hereby incorporated by reference herein in their entirety and for all purposes.

The preferred polyethylene glycol moieties are methoxy polyethylene glycol (mPEG) moieties. The moieties are preferably added using succinimidyl ester derivatives of the methoxy polyethylene glycol species. In one example a preferred succinimidyl ester derivative of a methoxy polyethylene glycol species includes: succinimidyl esters of carboxymethylated polyethylene glycol (SCM-PEG) of the following formula, 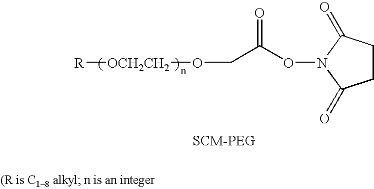
succinimidyl derivatives of poly(ethylene glycol) propionic acid (SPA-PEG) of the following formula, wherein R is C₁₋₈alkyl and n is an integer,
(R—(OCH₂CH₂)ₙ—O—CH₂CH₂—CO—OSu);
   and succinimidyl derivatives of poly(ethylene glycol) butanoic acid (SBA-PEG) of the following formula, wherein R is C₁₋₈alkyl and n is an integer,
(R—(OCH₂CH₂)ₙ—O—CH₂CH₂CH₂—CO—OSu).

Methods to prepare SCM-PEG, SPA-PEG, and SBA-PEG are well known in the art. For example, U.S. Pat. No. 5,672,662 to Harris et al. describes active esters of PEG acids and related polymers that have a single propionic or butanoic acid moiety and no other ester linkages. Preparation of SCM-PEG has been described in, for example, Veronese et al. (1989), Journal of Controlled Release, 110: 145-54.

The use of the term “SPA-PEG” includes mPEG-SPA (methoxy-PEG-Succinimidyl Propionate) and the use of the term “SBA-PEG” includes mPEG-SBA (methoxy-PEG-Succinimidyl Butanoate). Activated polymers such as SBA-PEG and SPA-PEG, are both commercially available and may be obtained from, e.g., Nektar, Inc., Huntsville, Ala., U.S.A.

The use of the term “SCM-PEG” (R—(OCH₂CH₂)ₙ—O—CH₂—CO—OSu; R is C₁₋₈alkyl and n is an integer) includes methoxy-PEG-succinimidyl ester of carboxymethylated PEG (mPEG-SCM). According to Greenwald et al., SCM-PEG “reaction with protein would form a stable amide, but t1/2 hydrolysis has been reported (Nektar, Huntsville, Ala., January 1996 catalog, p 46) as <1 min at pH 8, thus minimizing its usefulness for protein modification in aqueous solution . . . ” (Bioconjugate Chem., 7 (6), 638-641, 1996).

At present, SCM-PEG may be custom synthesized by, e.g., Delmar Chemicals, Inc, Quebec, Canada.

SCM-PEG, SPA-PEG and SBA-PEG react primarily with the primary amino groups of lysine and the N-terminal amino group. Reactions with EPO are shown below for SCM-PEG5K, SPA-PEG5K and SBA-PEG5K, respectively, wherein OSu represents n-hydroxysuccinimide, and m is 1-4, n is an integer:
(SCM-PEG) CH₃O—(OCH₂CH₂)N—O—CH₂—CO—OSU+EPO (NH₂)_{M} →CH₃O—(OCH₂CH₂)_{N}—O—CH₂—CO—NH-EPO
(SPA-PEG) CH₃O—(OCH₂CH₂)_{N}—O—CH₂CH₂—CO—OSU+EPO (NH₂)_{M} →CH₃O—(OCH₂CH₂)_{N}—O—CH₂CH₂—CO—NH-EPO
(SBA-PEG) CH₃O—(OCH₂CH₂)_{N}—O—CH₂CH₂CH₂—CO—OSU+EPO (NH₂)_{M} →CH₃O—(OCH₂CH₂)_{N}—O—CH₂CH₂CH₂—CO—NH-EPO

The combination dosing regimen of the present invention can be administered to any subject in whom an initial increase in total red blood cell count and/or hemoglobin concentration may be desired. Subjects may also benefit from the combination dosing regimen if maintenance, or management, of total red blood cell count and/or hemoglobin concentration is desired or required. That is, the combination dosage regimen may be administered prophylactically to provide an increase in, or to maintain or otherwise manage, total red blood cell count and/or hemoglobin in a subject before an event anticipated to impact total red blood cell count and/or hemoglobin concentration. Subjects that may benefit from the dosing regimen are not particularly limited and may include both human and animal subjects, and may preferably be mammalian subjects.

As discussed at length above, the combination dosing regimen described herein surprisingly and unexpectedly can not only stimulate production of reticulocytes, but also can sustain a significant portion of these reticulocytes as they develop into neocytes and ultimately, mature red blood cells. Further, this effect can be sustained by cycling the dosing segment in time increments greater than the conventional dosing of EPO would suggest. The dosing regimen is thus appropriately administered to any subject in need of such treatment, or to any subject anticipating a drop in total red blood cell count. Methods of treating a subject wherein an increased total red blood cell count and/hemoglobin concentration is required or desired, as well as for preventing a subject from experiencing an undesirable reduction in total red blood cell count and/or hemoglobin concentration, are thus also provided.

There are many situations or occurrences which may lead to a subject having a less than optimal red blood cell count and/or hemoglobin, and any of these may be appropriately treated, or substantially prevented, by the present method. Generally speaking, any condition or occurrence that may result in episodic or chronic tissue hypoxia is appropriately treated by the present method, and examples of these include physical exertion, travel to high altitude, loss of blood, improper diet, illness, or administration of certain other therapeutic agents such as chemotherapeutic agents, etc. Because the method of the present invention is so effective at increasing and sustaining total red blood cell count and/or hemoglobin concentration, it is expected to prove particularly beneficial when applied to subjects suffering from acute and/or chronic hypoxia, such as those subjects who have suffered a traumatic injury resulting in the loss of large amounts of blood, those patients undergoing chemotherapy, and the like.

Although EPO and methods of dosing the same, are readily commercially available, the convenience of administering the present dosing segment, or practicing the present methods, may be further enhanced via the provision of the components required to do so in the form of a kit, and such a kit is also contemplated to be within the scope of the present invention. Desirably, a kit for administering the dosing segment or practicing the present method would include at least a first dosing segment of EPO comprising a first dosing unit of EPO capable of providing an exposure to EPO effective to at least marginally increase production of reticulocytes. As mentioned above, the first exposure can comprise any dose in any desired dosing format, but may provide optimum results if provided in a fast-acting, bolus mode of administration, such as, e.g., syringe adapted for subcutaneous administration. A second dosing unit of EPO, comprising a different dosage amount and/or form of EPO than the first and capable of providing an exposure to EPO at least marginally effective to sustain the reticulocytes as they mature into red blood cells may also be advantageously provided. Similar to the first exposure, the second exposure may comprise any dose in any desired dosing format. However, and as also discussed above, optimum results may be provided if the second exposure is provided a format that provides a relatively sustained exposure to EPO, such as, e.g., multiple syringes adapted for daily low level subcutaneous doses, multiple oral dosage forms for daily administration, a long acting EPO in any dosing format, a transdermal patch, etc. Instructions could be included in any format, that would desirably indicate that results of the administration of the two dosing segments could be optimized if the second exposure is initiated within about 3 days but not more than about 10 days after the first exposure.

Any number of dosing segments may be provided in connection with the kit, with each dosing segment comprising the same or different dosages or dosing formats for each exposure, so that an individual kit may conveniently be provided for days, weeks, or months of treatment or prevention, as desired or required. However, in order to fully take advantage of the flexibility provided by the present combination dosing regimen, kits may desirably be provided comprising the components required to administer a single dosing segment, so that multiple kits, comprising any dosing segment, may be combined to provide the components to carry out any desired or required combination dosing regimen, as may be determined prior to treatment with the initial dosing segment, or after administration and analysis of the outcome thereof. The kits may even be further optimized for subjects having a variety of different etiologies. For example, a kit may be provided indicating that is particularly beneficial for the treatment chemotherapy patients, in which case, the instructions may indicate that the dosing segment provided therein is desirably repeated, and the repetition desirably initiated prior to each chemotherapy session.

The following examples are provided to illustrate the present invention, and should not be construed as limiting thereof. This invention will be better understood by reference to the schemes and examples that follow, but those skilled in the art will readily appreciate that these are only illustrative of the invention as described more fully in the claims which follow thereafter.

### COMPARATIVE EXAMPLE 1

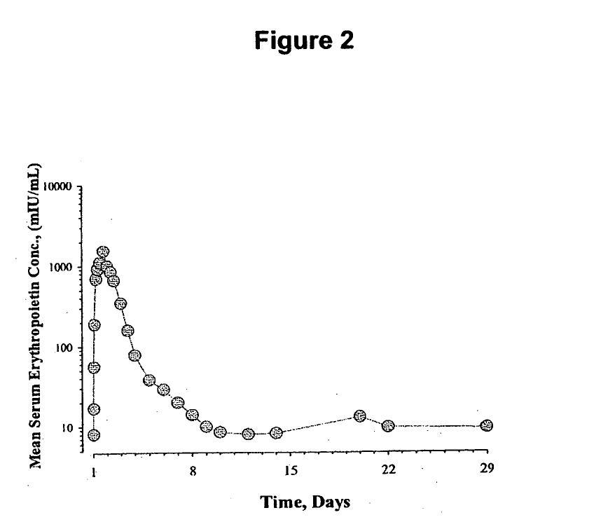

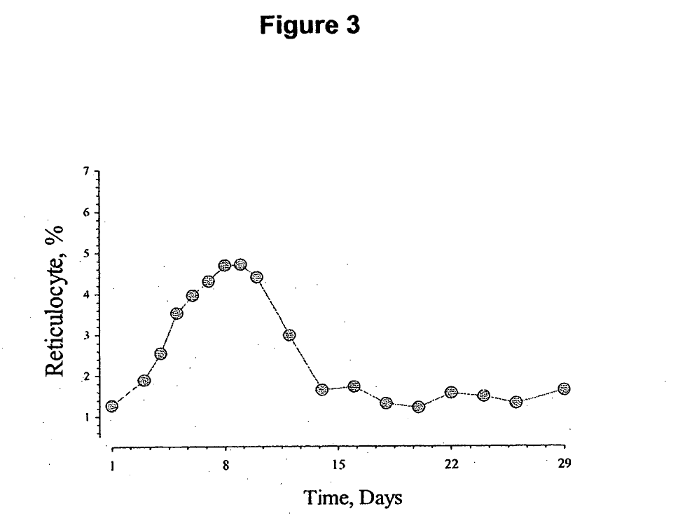

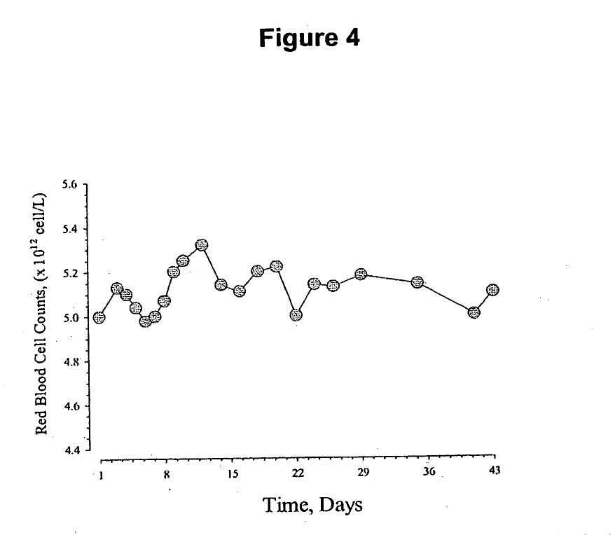

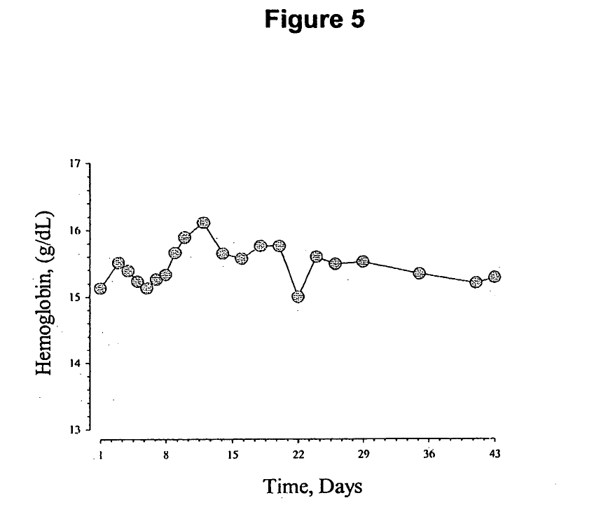

A single subcutaneous dose of 40,000 IU EPO (EPREX®, Ortho Biotech, Toronto, ON) was administered to healthy subjects (n=8). Serum samples were taken over 29 days and tested for EPO levels as well as for reticulocyte, hemoglobin and total red blood cell concentration. As shown in FIG. 2, serum EPO levels peaked upon dosing and then returned to endogenous levels in about 10 days. Further, as shown in FIG. 3, reticulocyte levels peaked approximately ten days post administration and returned to base line by Day 15. Finally, as shown in FIGS. 4 and 5, respectively, there was no sustained increase in total red blood cell count and hemoglobin concentration after a single subcutaneous administration of a typical therapeutic dose of EPO.

### EXAMPLE 1

Three groups of dogs (n=3 in each group) were used for this study. One group received a conventional dosing of EPO, i.e., a single 400 IU/kg subcutaneous dose on the first day of dosing, a second group received daily low level (25 IU/kg) subcutaneous doses of EPO on days 4-16 of testing, and a third group received a dosing segment in accordance with one embodiment of the present invention, i.e., a first exposure comprising a single 400 IU/kg subcutaneous dose of EPO on the first day of dosing, followed by a second exposure comprising daily 25 IU/kg subcutaneous doses of EPO on days 4-16 of testing. Serum samples were taken daily and tested for EPO, reticulocyte, and hemoglobin levels as well as total red blood cell count.

The results of this Example are shown in FIGS. 6-9, which clearly show that the dosing segment in accordance with this embodiment of the present invention produced a greater increase in both total red blood cell count and hemoglobin concentration than both conventional dosing as well as daily low level dosing. It is believed that, if this dosing segment were cycled after at least about 14 days, or even after up to at least about 32 days, the increase produced by the administration of the first dosing segment could be substantially maintained.

### EXAMPLE 2

Three groups of dogs (n=3 in each group) were used for this study. One group received a conventional dosing of EPO, i.e., a single 400 IU/kg subcutaneous dose on the first day of dosing, a second group received daily low level (50 IU/kg) subcutaneous doses of EPO on days 4-16 of testing, and a third group received a dosing segment in accordance with one embodiment of the present invention, i.e., a first exposure comprising a single 400 IU/kg subcutaneous dose of EPO on the first day of dosing, followed by a second exposure comprising daily 50 IU/kg subcutaneous doses of EPO on days 4-16 of testing. Serum samples were taken daily and tested for EPO, reticulocyte, and hemoglobin levels as well as total red blood cell count.

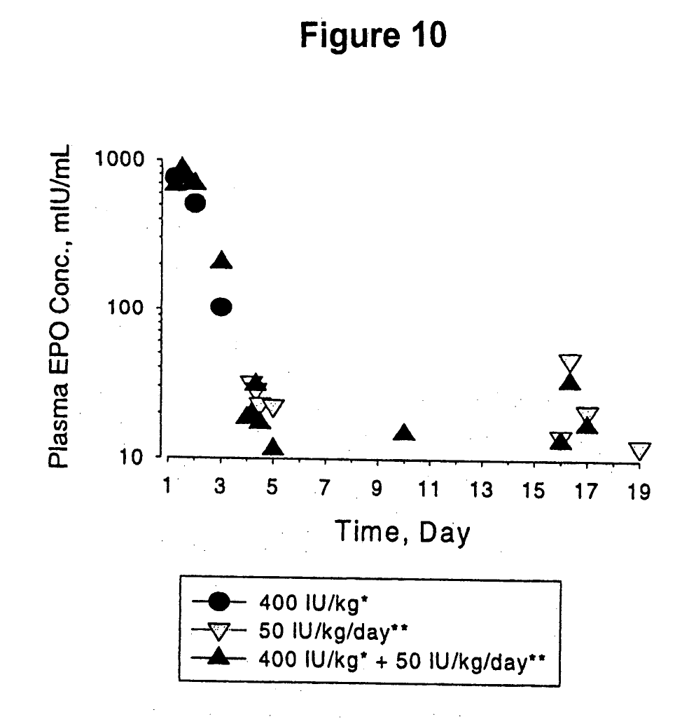

The results of this Example are shown in FIGS. 10-13, which clearly show that the dosing segment in accordance with this embodiment of the present invention produced a greater increase in both total red blood cell count and hemoglobin concentration than both conventional dosing as well as daily low level dosing. It is believed that, if this dosing segment were cycled after at least about 14 days, or even after up to at least about 32 days, the increase produced by the administration of the first dosing segment could be substantially maintained.

### EXAMPLE 3

Three groups of dogs (n=3 in each group) were used for this study. One group received a conventional dosing of EPO, i.e., a single 800 IU/kg subcutaneous dose on the first day of dosing, a second group received daily low level (25 IU/kg) subcutaneous doses of EPO on days 4-16 of testing, and a third group received a dosing segment in accordance with one embodiment of the present invention, i.e., a first exposure comprising a single 800 IU/kg subcutaneous dose of EPO on the first day of dosing, followed by a second exposure comprising daily 25 IU/kg subcutaneous doses of EPO on days 4-16 of testing. Serum samples were taken daily and tested for EPO, reticulocyte, and hemoglobin levels as well as total red blood cell count.

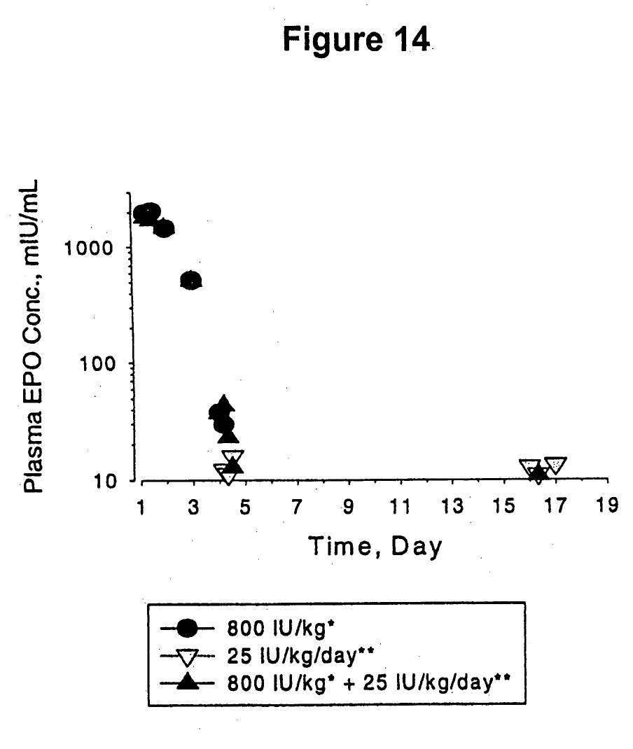

The results of this Example are shown in FIGS. 14-17, which clearly show that the dosing segment in accordance with this embodiment of the present invention produced a greater increase in both total red blood cell count and hemoglobin concentration than both conventional dosing as well as daily low level dosing. It is believed that, if this dosing segment were cycled after at least about 14 days, or even after up to at least about 32 days, the increase produced by the administration of the first dosing segment could be substantially maintained.

### EXAMPLE 4

Three groups of dogs (n=3 in each group) were used for this study. One group received a conventional dosing of EPO, i.e., a single 800 IU/kg subcutaneous dose on the first day of dosing, a second group received daily low level (50 IU/kg) subcutaneous doses of on days 4-16 of testing, and a third group received a dosing segment in accordance with one embodiment of the present invention, i.e., a first exposure comprising a single 800 IU/kg subcutaneous dose of EPO on the first day of dosing, followed by a second exposure comprising daily 50 IU/kg subcutaneous doses of EPO on days 4-16 of testing. Serum samples were taken daily and tested for EPO, retculocyte, and hemoglobin levels as well as total red blood cell count.

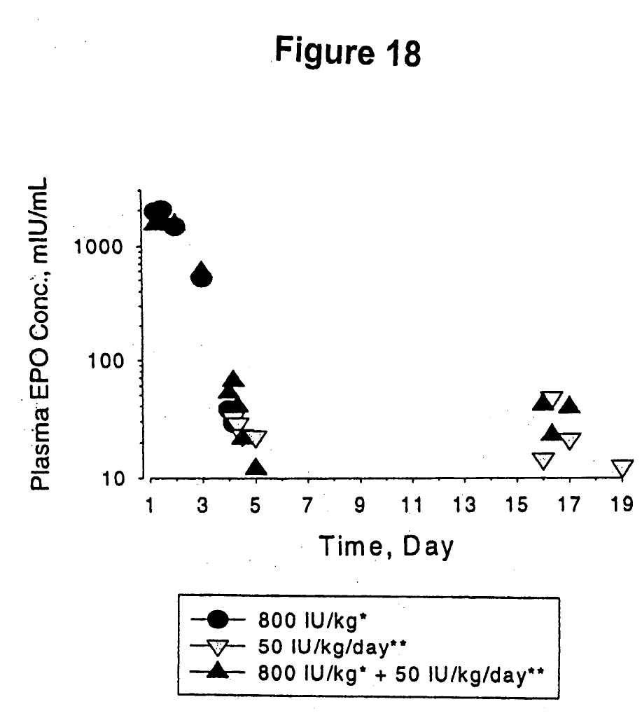

The results of this Example are shown in FIGS. 18-21, which clearly show that the dosing segment in accordance with this embodiment of the present invention produced a greater increase in both total red blood cell count and hemoglobin concentration than both conventional dosing as well as daily low level dosing. It is believed that, if this dosing segment were cycled after at least about 14 days, or even after up to at least about 32 days, the increase produced by the administration of the first dosing segment could be substantially maintained.

### EXAMPLE 5

Three treatment groups of dogs (n=3 in each group), all received conventional dosings of EPO (600 IU/kg/week) for 4 weeks. Group A then ceased dosing as a control group, while Group B received 1800 IU/kg EPO at week 5 and again at week 8, and Group C received a combination dosing regimen in accordance with the present invention comprising administration of a first dosing segment of a first exposure of 600 IU/kg of EPO on week 5 and a second exposure of 1200 IU/kg EPO on week 6, with the dosing segment being repeated in this Group on weeks 8 and 9, i.e., a first exposure of 600 IU/kg of EPO on week 8 and a second exposure of 1200 IU/kg on week 9. All groups had hemoglobin measured weekly.

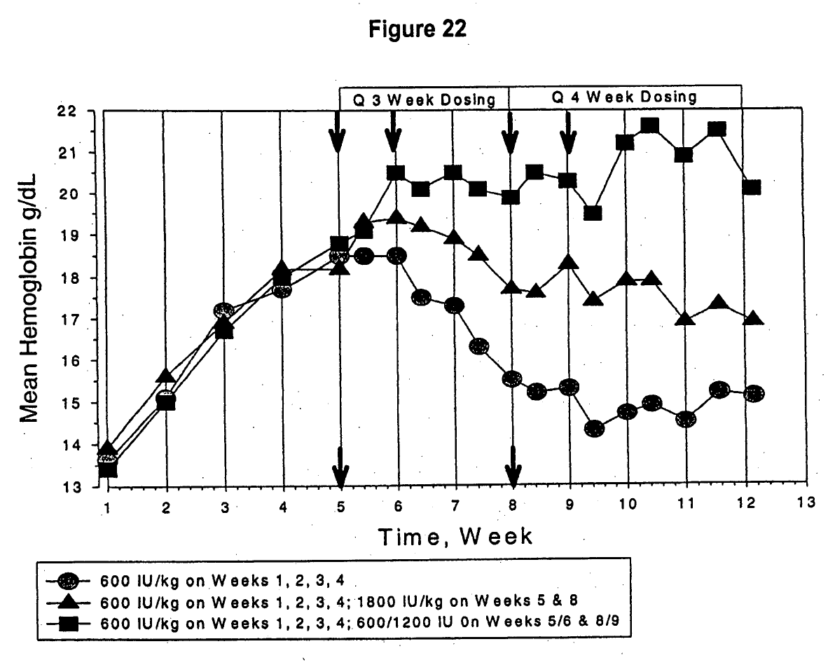

The results of this Example are shown in FIG. 22. As can be seen, hemoglobin levels in Group A declined naturally to endogenous levels even after conventional dosing of equivalent doses for 4 weeks. In contrast, hemoglobin was maintained in an elevated state for Group C, dosed in accordance with one embodiment of the inventive combination dosing regimen, not only between dosing segments, but also for at least about 4 weeks after the administration of the second exposure of the last dosing segment. Further, FIG. 22 shows that although Group B, which received the same total dose as Group C, maintained a higher level of hemoglobin than that of Group A, Group B did not maintain as high an elevation of hemoglobin as Group C, showing that the combination dosing regimen of the present invention provides a better hemoglobin response than administration of the same total dose via a different dosing regimen.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A combination dosing regimen of erythropoietin comprising administering at least a first dosing segment, said first dosing segment comprising a first exposure to EPO effective to increase production of reticulocytes followed by a second exposure to EPO effective to sustain the reticulocytes as they mature into red blood cells, wherein administration of the second exposure is initiated within about 3 days but not more than about 10 days after the first exposure.

2. The regimen of claim 1 wherein the dosing segment is repeated to provide a desired total red blood cell count and/or hemoglobin concentration over a desired time period.

3. The combination dosing regimen of claim 1, wherein the first exposure comprises a subcutaneous, intramuscular, intravenous or intra-peritoneal injection of EPO.

4. The combination dosing regimen of claim 3, wherein the second exposure comprises one or more daily subcutaneous injections of EPO of less than about 100 IU/kg.

5. The combination dosing regimen of claim 3, wherein the second exposure comprises the application of a transdermal patch comprising EPO.

6. The combination dosing regimen of claim 3, wherein the second exposure comprises the implantation of an implant comprising EPO.

7. The combination dosing regimen of claim 3, wherein the second exposure comprises the administration of a long-acting EPO.

8. The combination dosing regimen of claim 1, wherein the first exposure comprises a total dosing of EPO different than the second exposure.

9. The combination dosing regimen of claim 2, wherein the total red blood cell count and/or hemoglobin concentration is maintained between at least two dosing segments.

10. The combination dosing regimen of claim 9, wherein the dosing segment is repeated after at least about 8 weeks after the administration of the second exposure.

11. The combination dosing regimen of claim 10, wherein the dosing segment is repeated after at least about 4 weeks after the administration of the second exposure.

12. The combination dosing regimen of claim 11, wherein the dosing segment is repeated after at least about 2 weeks after the administration of the second exposure.

13. The combination dosing regimen of claim 1, wherein the dosing segment is repeated at least about 6 times.

14. The combination dosing regimen of claim 13, wherein the dosing segment is repeated at least about 4 times.

15. The combination dosing regimen of claim 14, wherein the dosing segment is repeated at least about 2 times.

16. A method for enhancing the production and maintenance of a desired mature red blood cell count and/or hemoglobin concentration comprising the step of administering at least a first dosing segment comprising a first exposure to EPO effective to increase production of reticulocytes followed by a second exposure to EPO effective to sustain the reticulocytes as they mature into red blood cells wherein administration of the second exposure is initiated within about 3 days but not more than about 10 days after the first exposure.

17. The method of claim 16, wherein the dosing segment may be repeated to provide a desired total red blood cell count and/or hemoglobin concentration over a desired time period.

18. The method of claim 16, wherein the method is utilized to treat a subject suffering from anemia.

19. The method of claim 18, wherein the anemia is chronic anemia.

20. The method of claim 16, wherein the method is utilized to treat a subject undergoing chemotherapy.

21. The method of claim 16, wherein the method is utilized to treat a subject that has suffered a traumatic injury.

22. The method of claim 16, wherein the first exposure comprises an EPO dose greater than the EPO dose of the second exposure.

23. The method of claim 16, wherein the first exposure comprises a subcutaneous injection of EPO.

24. The method of claim 16, wherein the second exposure comprises at least two daily subcutaneous injections of EPO of less than about 100 IU/kg.

25. The method of claim 16, wherein the second exposure comprises the application of a transdermal patch comprising EPO.

26. The method of claim 16, wherein the second exposure comprises the implantation of an implant comprising EPO.

27. The method of claim 16, wherein the second exposure comprises the administration of a long-acting EPO.

28. The method of claim 17, wherein the administration of subsequent dosing segments maintains the total red blood cell count and/or hemoglobin concentration produced by a preceding dosing segment.

29. The method of claim 28, wherein the dosing segment is administered at least about 8 weeks after the administration of the second exposure.

30. The method of claim 29, wherein the dosing segment is administered at least about 4 weeks after the administration of the second exposure.

31. The method of claim 30, wherein the dosing segment is administered at least about 2 weeks after the administration of the second exposure.

32. The method of claim 16, wherein the dosing segment is administered at least about 6 times.

33. The method of claim 32, wherein the dosing segment is administered at least about 4 times.

34. The method of claim 33, wherein the dosing segment is administered at least about 2 times.

35. A kit comprising at least a first dosing segment of EPO, said first dosing segment comprising a first dosing unit of EPO capable of providing an exposure to EPO effective to increase production of reticulocytes, a second dosing unit of EPO, comprising a dosage of EPO at a lower concentration than the first dosing segment and capable of providing an exposure to EPO effective to sustain the reticulocytes as they mature into red blood cells, and further comprising instructions indicating that administration of the second exposure is to be initiated at a time which is within about 3 days but not more than about 10 days after the first exposure.

36. The kit of claim 35, wherein the instructions further indicate that the dosing segment may be repeated.

37. The kit of claim 35, wherein the instructions further indicate that, for a subject undergoing chemotherapy, the dosing segment may desirably be repeated in concert with chemotherapy treatments.

38. The kit of claim 35, wherein the instructions indicate that the dosing segment is to be repeated after at least about 8 weeks after the administration of the second exposure.

39. The method of claim 38, wherein the instructions indicate that the dosing segment is to be repeated after at least about 4 weeks after the administration of the second exposure.

40. The method of claim 39, wherein the instructions indicate that the dosing segment is to be repeated after at least about 2 weeks after the administration of the second exposure.
